(19) United States
(12) Patent Application Publication
(10) Pub. No.: US 2023/0398267 A1
(43) Pub. Date: Dec. 14, 2023

(54) BREAST PUMP BAG

(71) Applicant: Raequael Patterson, Havre De Grace, MD (US)

(72) Inventor: Raequael Patterson, Havre De Grace, MD (US)

(21) Appl. No.: 18/140,609

(22) Filed: Apr. 27, 2023

Related U.S. Application Data
(60) Provisional application No. 63/336,268, filed on Apr. 28, 2022.

(51) Int. Cl. A61M 1/06

(52) U.S. Cl. CPC A61M 1/062

(57) ABSTRACT

A breast pump bag kit for carrying includes accessories used for breast milk collection. It includes a breast pump bag having an interior and an openable flap; a small bag which is adapted to be received with said interior of said breast pump bag; and a foldable pad which is adapted to be received with said interior of said breast pump bag. A removable pouch member is disposed inside said interior; and wherein in a second configuration said openable flap is in an opened position wherein said removable pouch member extends outside of said interior beyond said left side. A flexible fabric ring portion is disposed along a perimeter of said openable flap inside the interior, and is connected to the open end of the removable pouch by any of a zipper, snaps, and hook and loop faster. The bag has maximalist and minimalist configurations.

### CROSS REFERENCE TO RELATED APPLICATIONS

This application claims the priority of Provisional Application No. 63/336,268 filed on Apr. 28, 2022, inventor Raequael Patterson, entitled “Pump Caddy Organizer and the Vegan Leather Pump Bag”. The entire disclosure of this provisional patent application is hereby incorporated by reference thereto, in its entirety.

### STATEMENT REGARDING FEDERALLY SPONSORED RESEARCH OR DEVELOPMENT

Not applicable.

### FIELD OF THE INVENTION

The present invention relates to a breast pump bag. The present invention also relates to a kit for breast pumping including a breast pump bag, a small bag which is carried in the breast pump bag, and a foldable pad which is carried in the breast pump bag.

### BACKGROUND OF THE INVENTION

It is a problem in the art to provide a pump caddy that is useful, easy to carry, and affordable. It is also a problem in the art to provide a vegan leather pump bag that is useful, easy to carry, and affordable.

### SUMMARY OF THE INVENTION

From the foregoing, it is seen that it is a problem in the art to provide a device meeting the above requirements. According to the present invention, a device is provided which meets the aforementioned requirements and needs in the prior art. Specifically, the device according to the present invention provides a Breast Pump Bag.

The present invention also relates to a kit for breast pumping including a breast pump bag, a small bag which is carried in the breast pump bag, and a foldable pad which is carried in the breast pump bag.

The device of the present invention provides novel features and construction, and particularly provides an additional strap conveniently located for use.

The Breast Pump Bag according to the present invention has a minimalist configuration and a maximalist configuration.

Other objects and advantages of the present invention will be more readily apparent from the following detailed description when read in conjunction with the accompanying drawings.

### BRIEF DESCRIPTION OF THE DRAWINGS

FIG. 1 is an exploded view of a Breast Pump Bag kit, including a Breast Pump Bag, a small bag which is carried in the breast pump bag, and a foldable pad which is carried in the breast pump bag. In FIG. 1, the Breast Pump Bag is shown as a schematic front view, the smaller bag is shown in front perspective view viewed from the front and side, and the foldable bag is shown in a front view.

FIG. 2 is a perspective view of a rear side of the Breast Pump Bag of FIG. 1.

FIG. 3 is a schematic front view of a removable pouch member for use in the Breast Pump Bag of FIG. 1 and FIG. 2.

FIG. 4 is a perspective end view of the removable pouch member of FIG. 3.

FIG. 5 is a front view of the smaller bag of FIG. 1, schematically shown with its top partially opened.

FIG. 6 is a perspective view of the foldable pad of FIG. 1, shown partially unfolded.

FIG. 7 is a perspective view of the foldable pad of FIG. 1 and FIG. 6, shown in a completely unfolded configuration.

FIG. 8 is a front perspective view of the foldable pad of FIG. 6 and FIG. 7, viewed from the front and slight left point of view.

FIG. 9 is a schematic top view of the foldable pad of FIG. 8, showing the manner of folding of the folded portions, and omitting other details for the sake of clarity.

FIG. 10 is a perspective rear view of the Breast Pump Bag of FIG. 2 in a maximalist configuration showing the removable pouch member (shown in FIG. 4) and viewed from the front and slightly above and slightly to the right.

FIG. 11 is a perspective left view of the Breast Pump Bag of FIG. 1 viewed slightly above and toward the rear, with a flap open showing an interior of the Breast Pump Bag including a flexible fabric ring portion. In this view, the removable pouch member is removed and shown on the left side of this figure, and can be attached (by snaps, a zipper, or a hook-and-loop fastener such as Velcro®) to an attachment portion shown in the opening of the Breast Pump Bag.

FIG. 12 is a greatly enlarged view of a portion of the flexible fabric ring portion of FIG. 11, but with a zipper for attaching the removable pouch member, as another embodiment of the invention. In this case, the removable pouch member with have a corresponding zipper attachment means for cooperating with the zipper of the flexible fabric ring portion of FIG. 11.

FIG. 13 is a schematic sectional view of the flexible fabric ring portion of FIG. 12 taken along line 13-13 of FIG. 12, schematically showing the zipper feature.

FIG. 14 is a schematic top view of the Breast Pump Bag of FIG. 1, showing pockets in an interior portion of the Breast Pump Bag.

FIG. 15 is a schematic view of an interior side of a front wall of the Breast Pump Bag of FIG. 1, showing two pockets formed in the interior of the front wall.

FIG. 16 is a schematic view of an interior side of a rear wall of the Breast Pump Bag of FIG. 1, showing one pocket formed in the interior of the rear wall.

FIG. 17 is a schematic front view of a further embodiment of the removable pouch member for of FIG. 3 and FIG. 11, wherein it can be attached to the zipper feature of FIG. 12 and FIG. 13. In this view, the zipper feature replaces the snap feature of FIG. 3 and FIG. 11.

### DETAILED DESCRIPTION OF THE INVENTION

The present invention relates to a Breast Pump Bag 100, and also relates to a kit for breast pumping including the breast pump bag 100, a small bag 10 which is carried in the breast pump bag 100, and a foldable pad 20 which is carried in the breast pump bag 100. The device of the present invention provides novel features and construction, and can be formed of any fabric material used for bags and preferably is formed of Vegan leather.

A typical breast pump kit, hereafter referred to as a collection kit, includes flanges, valves, diaphragms, tubing, an AC adapter, and batteries, along with storage bags or storage containers with lids. Clean pump bottles are also needed, preferably one for each pumping session plus an additional one, including lids. A cooler bag and ice packs are also preferably included for storing and transporting the expressed breast milk. Further, wet hand wipes are preferably included, as well as dry wipes.

Nursing pads also should be included in the kit. Optional items include nipple, cream and shields.

An electrical breast pump can be used, and manually operated breast pumps are also available.

The Breast Pump Bag 100 according to the present invention has a minimalist configuration and a maximalist configuration, as shown in the attached drawings and as described below.

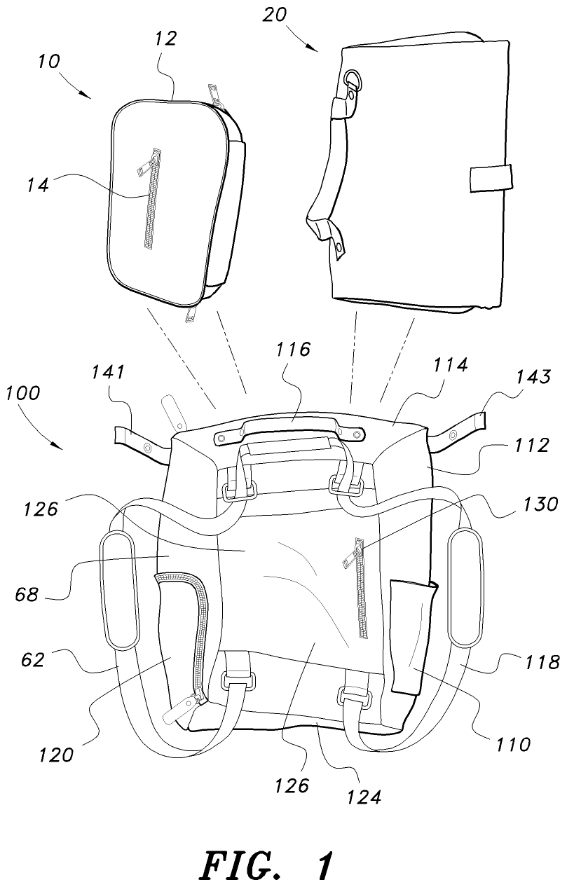

FIG. 1 is an exploded view of a Breast Pump Bag kit, including a Breast Pump Bag 100, a small bag 10 which is carried in the breast pump bag 100, and a foldable pad 20 which is carried in the breast pump bag 100. In FIG. 1, the Breast Pump Bag 100 is shown as a schematic front view, the smaller bag 10 is shown in front perspective view viewed from the front and side, and the foldable bag 20 is shown in a front view.

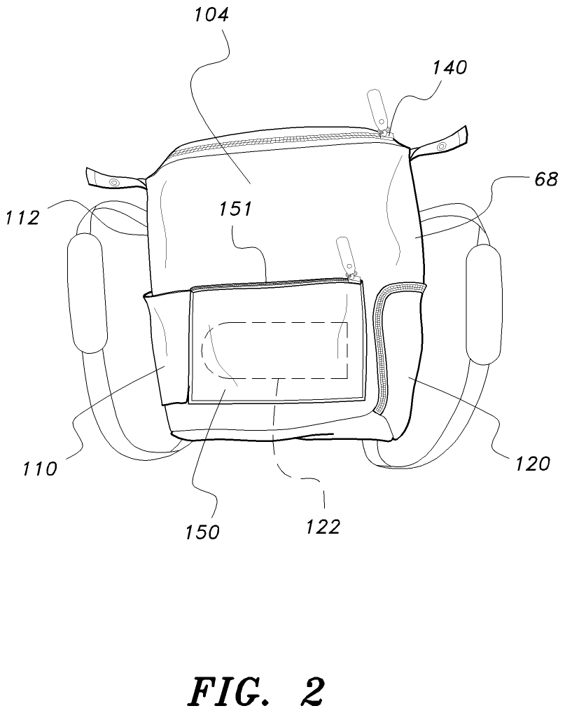

As shown in FIG. 1, the breast pump bag 100 has a top end 114, a right side 112, a right outside pocket 110, a bottom side 124, a left side 68, a rear side 104 (shown in FIG. 2), and a front side 126. The breast pump bag 100 includes a strap 141 on its left side near the top of the breast pump bag 100, and a strap 143 on its right side near the top of the breast pump bag 100; and also shows a top handle strap 116, and a pair of shoulder straps 118 and 62 threaded through loops (unnumbered in FIG. 1) and having shoulder support pads for comfortable use by a user carrying the breast pump bag 100 like a backpack. The straps 118 and 62 and associated loops are conventional elements in the backpack art, and all variations and other types of straps and support members can be used within the ambit of skill of any one having skill in the backpack and luggage arts and are contemplated as being within the scope of the present invention.

All variations in materials and hardware associated with the bag 100, the smaller bag 10, and the foldable member 20, and associated hardware elements within the ambit of skill of any one having skill in the backpack and luggage arts, are contemplated as being within the scope of the present invention.

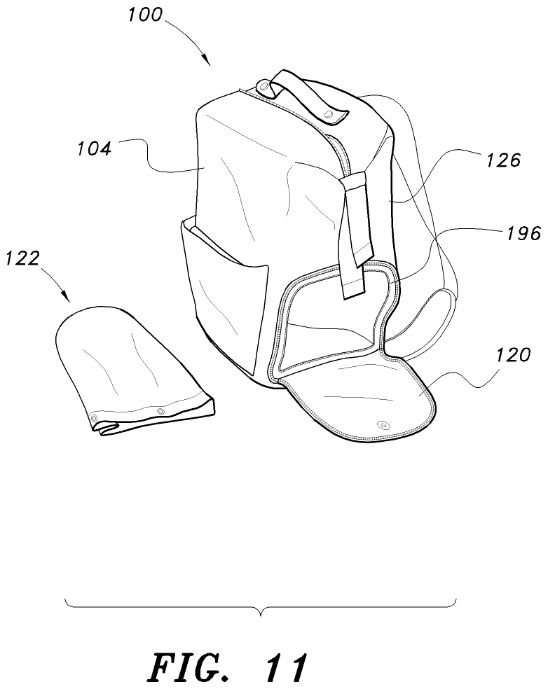

An openable flap 120 is shown in FIG. 11 and is formed in the left side 68, and an outer zipper pocket 130 is disposed in the front side 126. The pocket 130 is formed in a conventional way, and all variations within the ambit of skill of any one having skill in the backpack and luggage arts are contemplated as being within the scope of the present invention. The top end 114 has a zipper closure member 140, not visible in this view and is shown in FIG. 2. The zipper 140 is a conventional feature, and other closure variations are possible and all are contemplated as being within the scope of the present invention.

FIG. 2 is a perspective view of a rear side of the Breast Pump Bag 100 of FIG. 1. The bag 100 includes a zippered pouch 150 having a zipper closure 151. The bag 100 also includes a rear wall 104, and the top zipper 140 for closing and opening the bag 100.

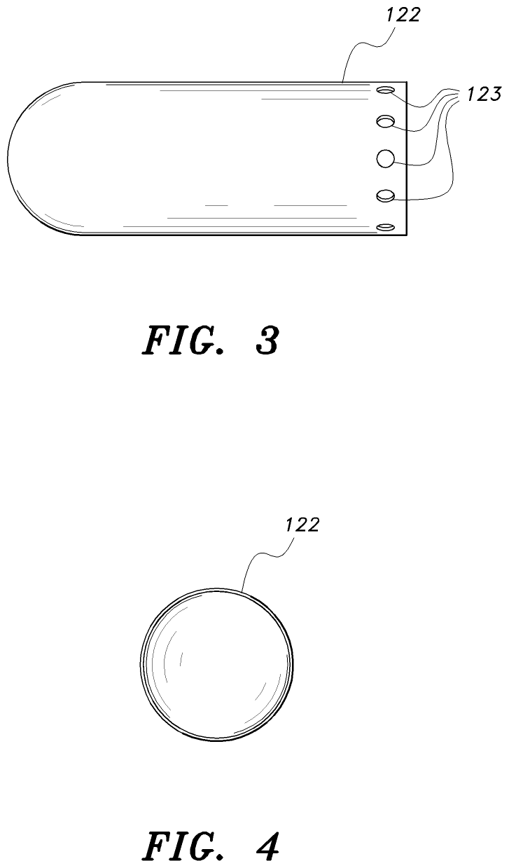

A removable pouch member 122 is schematically shown in dashed outline inside the breast pump bag 100, and is shown in detail in FIG. 3 and FIG. 4, discussed further below. FIG. 2 shows a minimalist configuration of the pump bag 100, wherein the removable pouch member 122 extends into an interior of the pump bag 100. The removable pouch member 122 can additionally be separated as shown in FIG. 11 and then placed inside the pump bag 100 loosely, for future use.

FIG. 3 is a schematic front view of the removable pouch member 122 for use in the Breast Pump Bag 100 of FIG. 1 and FIG. 2. In the view, a plurality of receiving elements 123 are shown for receiving snaps of corresponding snap fasteners of the bag 100 as discussed further below. Alternatively, the male portions of snap fasteners can be provided on the pouch member 122, with corresponding female portions disposed on the bag 100. Velcro® can be used instead of the elements 123, which is a brand name of one type of hook-and-loop fastener.

FIG. 4 is a perspective end view of the removable pouch member 122 of FIG. 3. This view shows that the pouch has an empty interior. It is shown in a rounded configuration for the sake of clarity; in use the pouch member is flexible and will lie flat as shown in FIG. 11.

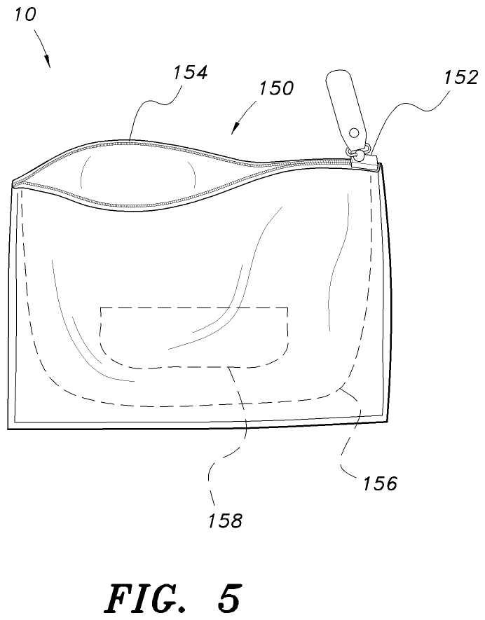

FIG. 5 is a front view of the smaller bag 10 of FIG. 1, schematically shown with a top portion 150 partially opened. The smaller bag 10 has a zippered closure 152 and zipper edges 154. The smaller bag 10 includes an interior pouch 158, which is covered by a larger mesh pouch 156 shown in dashed outline.

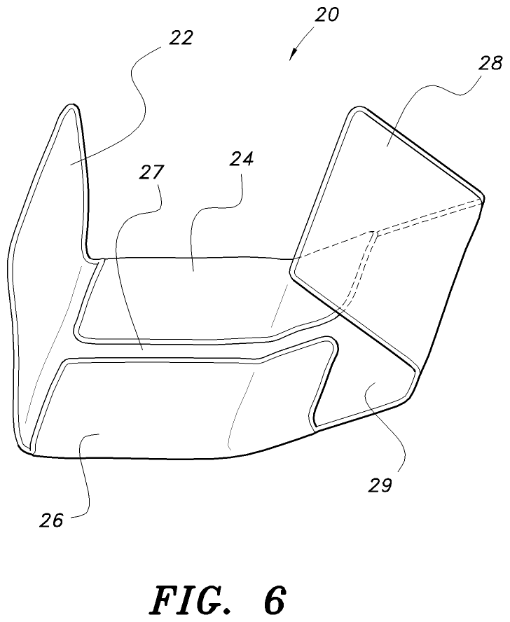

FIG. 6 is a perspective view of the foldable pad 20 of FIG. 1, shown partially unfolded. The foldable pad 20 includes opposed flap portions 24 and 26 which both are attached to and are folded over a central pad portion 27. A bottom pad portion 22 also extends from the central pad portion 27. An upper pad portion 29 is foldably connected to the central pad portion 27, and a top pad portion 28 is foldably connected to the upper pad portion 29.

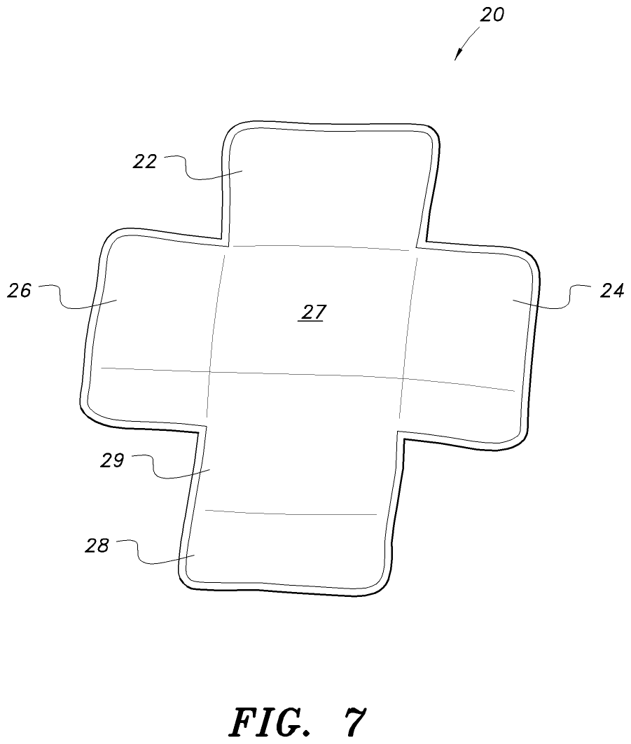

FIG. 7 is a perspective view of the foldable pad 20 of FIG. 1 and FIG. 6, shown in a completely unfolded configuration. In this view, the foldable pad 20 lies flat.

FIG. 8 is a front perspective view of the foldable pad 20 of FIG. 6 and FIG. 7, viewed from the front and slight left point of view. In this view, the foldable pad 20 is held in a folded configuration by a strap element 21 having a snap fastener (not numbered).

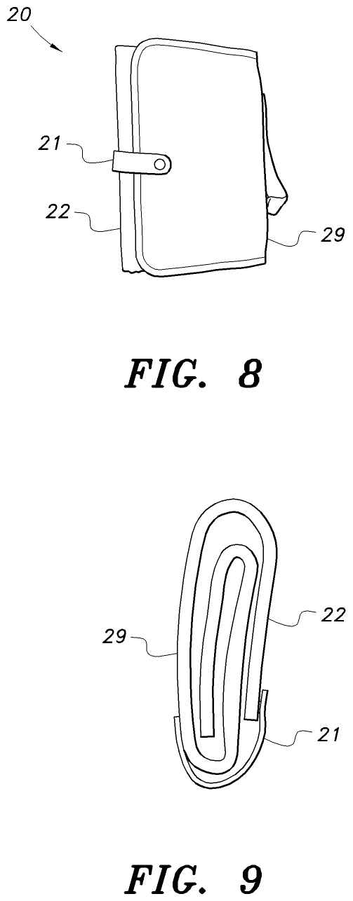

FIG. 9 is a schematic top view of the foldable pad 20 of FIG. 8, showing the manner of folding of the foldable portions while in the folded configuration. This view is a simplified view and omits other details for the sake of clarity.

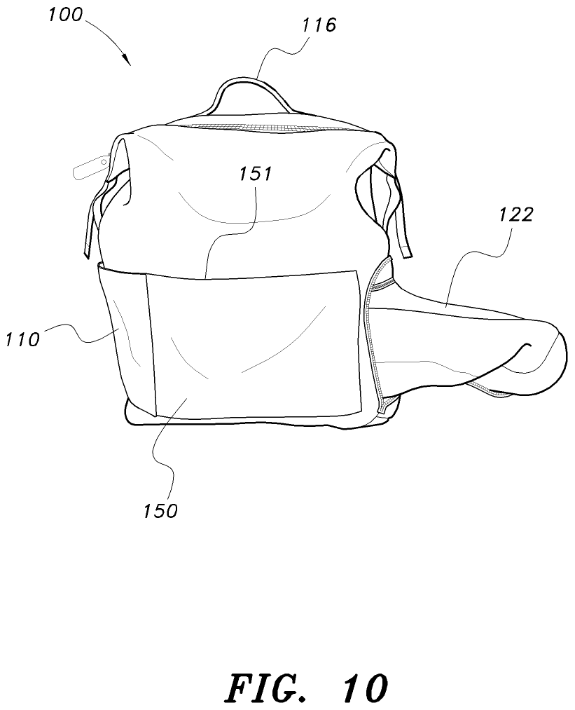

FIG. 10 is a perspective rear view of the Breast Pump Bag 100 of FIG. 2 in a maximalist configuration, showing the removable pouch member 122 (shown in FIG. 4) and viewed from the front and slightly above and slightly to the right. Here, the removable pouch member 122 is fastened at one end to the interior of the bag 100, and extends outwardly, creating a large amount of interior room. The interior room created by this configuration is adapted to contain breast pump items such as a manual or electric breast pump (not shown).

FIG. 11 is a perspective left view of the Breast Pump Bag 100 of FIG. 1 viewed slightly above and toward the rear, with the flap 120 open showing an interior of the Breast Pump Bag including a flexible fabric ring portion 196. The ring portion 126 is shown pulled outwardly so that it is in true view in this figure, and when not in use would tend to like flat against the interior sides of the bag 100. The ring portion 126 carries fastener elements of the type described hereinabove, such as snap fasteners. In this view, the removable pouch member 122 is removed and is shown on the left side of this figure in a flaccid configuration, and the member 122 can be attached to the ring portion 126 by snaps, a zipper, or a hook-and-loop fastener such as Velcro® and all such variations are contemplated as being within the scope of the present invention.

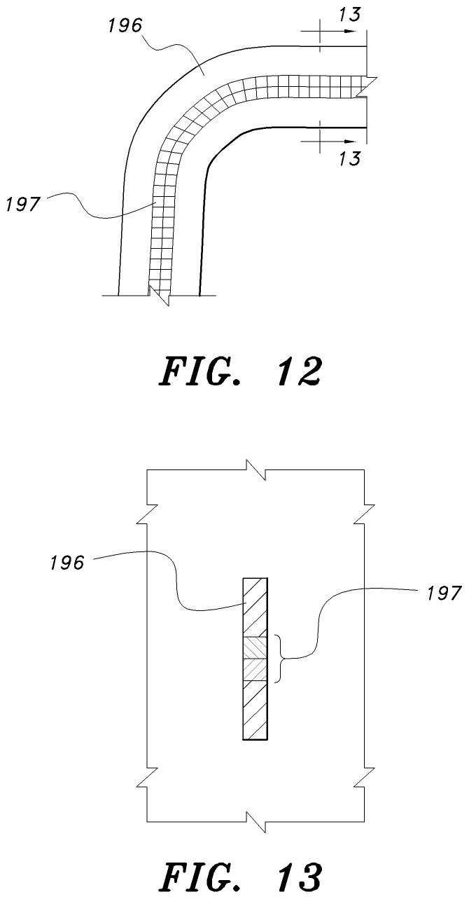

FIG. 12 is a greatly enlarged view of a portion of the flexible fabric ring portion of FIG. 11, but with a zipper 197 for attaching the removable pouch member 122, as another embodiment of the invention. The zipper 197 is bordered by supporting thin fabric material 196. In this case, the removable pouch member with have a corresponding zipper attachment means for cooperating with the zipper of the flexible fabric ring portion of FIG. 11.

FIG. 13 is a schematic sectional view of the flexible fabric ring portion of FIG. 12 taken along line 13-13 of FIG. 12, schematically showing the zipper feature 197 and supporting fabric 196.

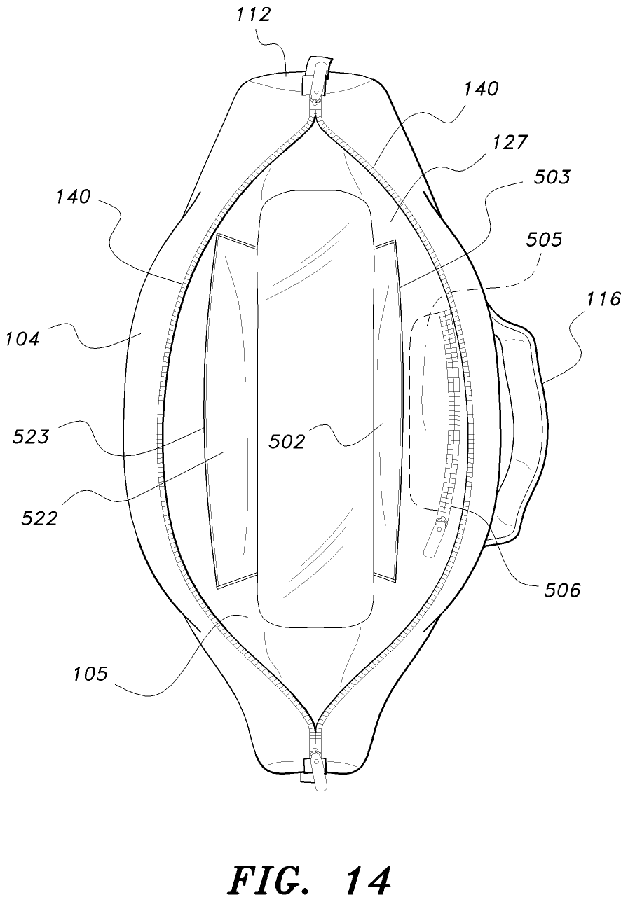

FIG. 14 is a schematic top view of the Breast Pump Bag 100 of FIG. 1 in an open position showing an interior of the Breast Pump Bag 100, showing inner side walls 105 and 127, the top zipper 140, a pocket 522, a pocket 502, a zipper 506, a top edge 523, and the handle 116. pockets in an interior portion of the Breast Pump Bag 100. This view is schematic.

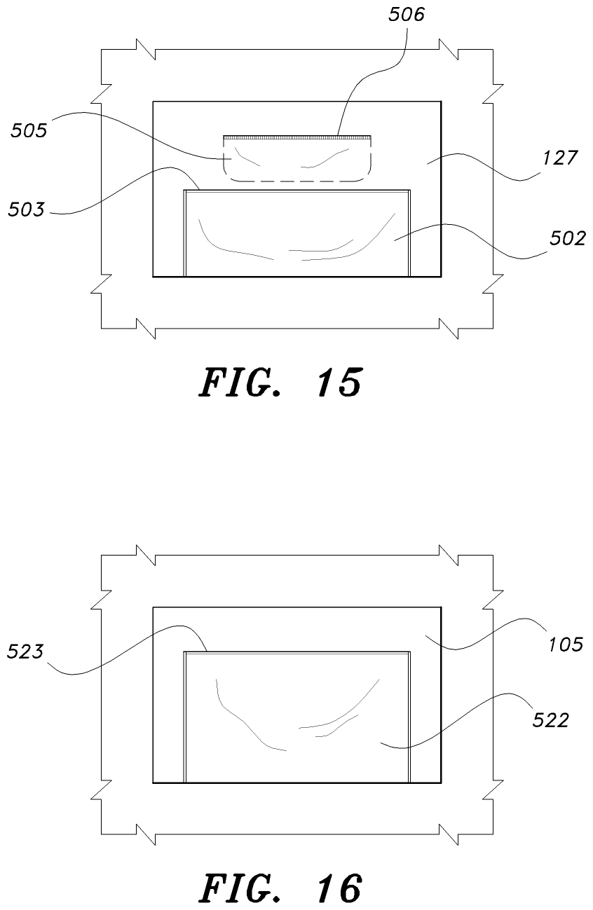

FIG. 15 is a schematic view of an interior side of a front wall 127 of the Breast Pump Bag 100 of FIG. 1, showing two pockets formed in the interior of the front wall 127. Specifically the pocket 502 is shown having a top edge 503 serving as an opening to the pocket 502, and the pocket 505 is shown having a zipper 506 serving as an opening to the pocket 505.

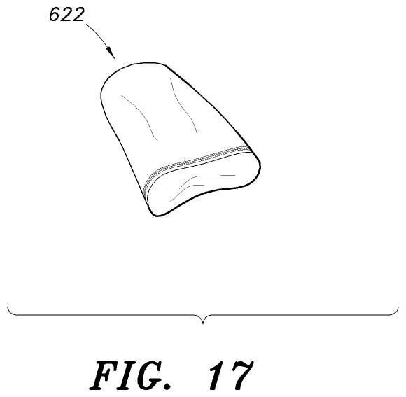

FIG. 16 is a schematic view of an interior side of the rear wall 105 of the Breast Pump Bag 100 of FIG. 1, showing one pocket 522 formed in the interior of the rear wall 105, and having a top edge 523 serving as an opening to the pocket 522..

FIG. 17 is a schematic front view of a further embodiment of the removable pouch member of FIG. 3 and FIG. 11, namely pouch 622 having a zipper feature at its open end, for attachment to the thin fabric portion carrying the zipper 197 feature of FIG. 12 and FIG. 13. In this view, the zipper feature at the end of the pouch 622 replaces the snap feature of FIG. 3 and FIG. 11.

The invention being thus described, it will be evident that the same may be varied in many ways by a routineer in the applicable arts. Such variations are not to be regarded as a departure from the spirit and scope of the invention and all such modifications are intended to be included within the scope of the claims.

What is claimed is:

1. A breast pump bag kit for carrying includes accessories used for breast milk collection, comprising:
a breast pump bag having an interior and an openable flap;
a small bag which is adapted to be received with said interior of said breast pump bag; and
a foldable pad which is adapted to be received with said interior of said breast pump bag.

2. The breast pump bag kit of claim 1, where said breast pump bag comprises:
a top end,
a bottom side,
a front side,
a rear side,
a right side,
a left side having an openable flap,
an interior bounded by said top end, said bottom side, said front side, said rear side, and said left side, and
a removable pouch member;
wherein in a first configuration said openable flap is in a closed position and said removable pouch member is disposed inside said interior; and wherein in a second configuration said openable flap is in an opened position wherein said removable pouch member extends outside of said interior beyond said left side.

3. The breast pump bag kit as claimed in claim 2, wherein said removable pouch member has a tubular form, having one open end.

4. The breast pump bag kit as claimed in claim 2, wherein said open end of said removable pouch member has a connecting means so that it can be connected to said left side of said breast pump bag.

5. The breast pump bag kit as claimed in claim 4, wherein said connecting means of said open end of said removable pouch member comprises a zipper.

6. The breast pump bag kit as claimed in claim 4, wherein said connecting means of said open end of said removable pouch member comprises snap members.

7. The breast pump bag kit as claimed in claim 4, further comprising a flexible fabric ring portion connected along a perimeter of said openable flap along said interior of said breast pump bag, said flexible fabric ring portion being connectable to said connecting means of said open end of said removable pouch member.

8. The breast pump bag kit as claimed in claim 7, wherein said connecting means of said open end of said removable pouch member comprises a zipper.

9. The breast pump bag kit as claimed in claim 1, wherein said breast pump kit includes accessories used for breast milk collection.

10. The breast pump kit as claimed in claim 9, wherein said accessories include any of:
flanges, valves, diaphragms, tubing, an AC adapter, batteries, storage bags, dry wipes, wet hand wipes, storage containers with lids, and clean pump bottles.

11. A breast pump bag for carrying includes accessories used for breast milk collection, comprising:
a breast pump bag body having an interior and an openable flap.

12. The breast pump bag of claim 11, where said breast pump bag body comprises:
a top end,
a bottom side,
a front side,
a rear side,
a right side,
a left side having an openable flap,
an interior bounded by said top end, said bottom side, said front side, said rear side, and said left side, and
a removable pouch member;
wherein in a first configuration said openable flap is in a closed position and said removable pouch member is disposed inside said interior; and wherein in a second configuration said openable flap is in an opened position wherein said removable pouch member extends outside of said interior beyond said left side.

13. The breast pump bag as claimed in claim 11, wherein said removable pouch member has a tubular form, having one open end.

14. The breast pump bag as claimed in claim 13, wherein said open end of said removable pouch member has a connecting means so that it can be connected to said left side of said breast pump bag.

15. The breast pump bag as claimed in claim 14, wherein said connecting means of said open end of said removable pouch member comprises a zipper.

16. The breast pump bag as claimed in claim 14, wherein said connecting means of said open end of said removable pouch member comprises snap members.

17. The breast pump bag as claimed in claim 14, further comprising a flexible fabric ring portion connected along a perimeter of said openable flap along said interior of said breast pump bag, said flexible fabric ring portion being connectable to said connecting means of said open end of said removable pouch member.

18. The breast pump bag as claimed in claim 17, wherein said connecting means of said open end of said removable pouch member comprises a zipper.
